(19) United States
(12) Patent Application Publication
(10) Pub. No.: US 2024/0117562 A1
(43) Pub. Date: Apr. 11, 2024

(54) Compositions for Improving the Environmental Impact of Textiles and Leather

(71) Applicant: Locus Solutions IPCo, LLC, Solon, OH (US)

(72) Inventors: Sean FARMER, Ft. Lauderdale, FL (US); Ken ALIBEK, Solon, OH (US); Andrew R. LEFKOWITZ, Solon, OH (US)

(21) Appl. No.: 18/010,569

(22) PCT Filed: Aug. 11, 2022
(86) PCT No.: PCT/US2022/040088

§ 371 Date: Feb. 16, 2023

Related U.S. Application Data
(60) Provisional application No. 63/231,820, filed on Aug. 11, 2021.

(51) Int. Cl. D06M 13/224; C12P 7/6481; C12P 19/44; C14C 11/00

(52) U.S. Cl. CPC D06M 13/2246; C12P 7/6481; C12P 19/44; C14C 11/00

(57) ABSTRACT

The subject invention provides environmentally-friendly compositions and methods for enhancing production of textiles and leather. In certain embodiments, the methods of the subject invention comprise incorporating the application of a biological amphiphilic molecule into a textile or leather-making process to reduce chemical usage, reduce water usage, reduce water pollution and/or provide an added benefit to the process. In certain embodiments, the methods of the subject invention comprise substituting a chemical surfactant with a biological amphiphilic molecule in one or more steps involved in textile or leather-making process that would traditionally utilize a chemical surfactant.

### CROSS-REFERENCE TO RELATED APPLICATION

This application claims priority to U.S. Provisional Patent Application No. 63/231,820, filed Aug. 11, 2021, which is incorporated herein by reference in its entirety.

### BACKGROUND OF THE INVENTION

Surfactants are surface-active, amphiphilic molecules with potential applications in many areas of industry. Accordingly, the market for surfactants, which currently consists of thousands of different surface-active molecules, is growing rapidly. About 60% of surfactants are used as detergents and compounds for personal care products. Other uses include, for example, pharmaceuticals and supplements; oil and gas recovery; bioremediation; agriculture; cosmetics; coatings and paints; food production and processing; and construction.

The properties of a surface-active molecule can be measured by hydrophile-lipophile balance (HLB). HLB is the balance of the size and strength of the hydrophilic and lipophilic moieties of a surface-active molecule. Specific HLB values are required to, for example, form a stable emulsion. In water/oil and oil/water emulsions, the polar moiety of the surface-active molecule orients towards the water, and the non-polar group orients towards the oil, thus lowering the interfacial tension between the oil and water phases.

HLB values range from 0 to about 20, with lower HLB (e.g., 10 or less) being more oil-soluble and suitable for water-in-oil emulsions, and higher HLB (e.g., 10 or more) being more water-soluble and suitable for oil-in-water emulsions. Other properties, such as foaming, wetting, detergency and solubilizing capabilities, are also dependent upon HLB.

Synthetic and chemical surfactants are advantageous because they can be easy to produce and can be tailored to perform a desired function based on their molecular structure. Thus, thousands of different surfactants have been developed, each having a certain narrow function. While this leaves ample options to choose from when producing products in which surfactants are used, the specificity of surfactant functions means that more varieties and combinations of surfactants are required for producing products with multiple functions. For example, a surfactant useful as a wetting agent may not necessarily be useful as a detergent, and a surfactant useful as an emulsifier may not necessarily be useful as an anti-corrosion agent.

The result is the over-use and over-production of chemical surfactants over the course of many decades. With growing consumer and regulatory awareness, the shortcomings of chemical surfactants are beginning to surface, including, for example, their potential and known toxicity to humans and animals; persistence in the environment, including aquatic environments, soil and ground water; contribution to climate change during production and application; and incompatibility with other chemicals.

Two examples of industries with high environmental impacts that utilize surfactants include textile production and leather-making. These industries involve water- and energy-intensive processes combined with heavy chemical and surfactant usage, which lead to concerns such as health hazards and wastewater pollution.

Textiles are flexible sheets of material made from the weaving of yarns or threads comprising naturally-derived fibers, such as plant matter, animal hair, and/or minerals, or synthetic fibers, such as polyesters. Examples of textiles include fabric and cloth used for making clothing, upholstery and drapes, as well as carpeting and rugs.

Fibers often contain greasy or waxy hydrophobic impurities. These impurities can create barriers to further steps in the treatment and processing of textiles. Thus, chemical surfactants are often used to scour, or clean, the fibers prior to further processing. Because scouring can be drying to the fibers, lubrication is necessary prior to spinning, cording, and weaving the fibers into textiles. Again, chemical surfactants are often employed in this step as emulsifiers for lubricating agents. Finally, before and/or after weaving, textiles are dyed and finished. This step involves the largest use of surfactants in the textile industry, namely as wetting agents to allow the dyes, inks and finishing materials (e.g., antimicrobials, fire retardants, antistatics, etc.) to penetrate the fibers. Polymeric binders are also used to ensure strength of color and fastness of pigmented ink on textiles.

Ethoxylates (e.g., fatty alcohol ethoxylates, fatty acid ethoxylates and fatty amine ethoxylates) and sulfates (e.g., alkyl sulfates and sulfonates, aryl sulfates and sulfonates, and a variety of sulfosuccinate surfactants) are common surfactants utilized in scouring, lubrication and dyeing and finishing textiles. While these chemicals are low cost and multi-functional, they can become wastewater pollutants, such as 1,4-dioxane. Additionally, they have a high critical micelle concentration (CMC), meaning significant quantities are needed for achieving desired performance. Thus, processing of industrial textiles often involves the large use of chemicals, including surfactants, which can, in turn, be a large source of low-biodegradability water pollutants.

Similarly to textiles, leather making also involves heavy use of chemicals, including surfactants. The first aspect of leather making is to irreversibly stabilize a hide that is otherwise prone to putrefaction, and the second is to impart desirable properties on the leather for final use.

Hides and skins usually arrive in a tannery in a temporarily cured state, usually brought about by packing in salt or by chilling. The first process in the tannery is then to soak the cured hides in clean water to remove salt, blood, dung and other dirt. Soaking also ensures complete rehydration of the hides in preparation for fleshing. The soaked hide is then passed through a fleshing machine that mechanically removes flesh or fat deposits from the flesh side of the hide, thereby facilitating chemical treatment in subsequent processes.

In order to remove hair, epidermis and residual inter-fibrillary components from the hide, it is soaked up to about 24 hours in a solution containing lime and sodium sulfide or other alkali materials. This step of liming also serves to open up the fiber structure of the hide. The hide is then de-limed by soaking in a weak acid solution of, for example, ammonium sulfate and ammonium chloride. This solution reacts with mechanically or chemically bound lime to form water-soluble salts, which can then be washed from the hide to remove the lime. Most types of hide are simultaneously treated with a bating material consisting of enzymes, usually pancreatic enzymes, which are used to remove residual components broken down by the lime. The bating material has the added benefit of giving a smoother grain and rendering the hide soft and flexible.

After de-liming and bating, the hides are washed in water several times to prepare them for pickling to acidify the hides prior to tanning. Metal salts such as chromium salts, aluminum salts or zirconium salts or mixtures thereof are often use in tanning. Alternatively, the hides may be pre-tanned after the pickling stage, using for example modified aldehydes such as glutaraldehyde and modified glutaraldehyde. Natural vegetable tanning agents or synthetic organic tanning agents may also be used.

Hides may then be processed further using, for example, additional tanning, polymeric materials, dyes and fat liquors to impart, for example, specific properties such as color, water-repellence and softness.

Dying is a particularly important part of leather processing, and it is frequently the most expensive single treatment. A variety of dyes may be used in the dying of leather, including acid dyes, direct dyes, solubilized sulfur dyes and reactive dyes. Common problems in leather dyeing include lack of uniformity and lack of penetration.

Fat liquoring is also an important process. In general, fatliquors include oils, fats, and waxes from natural or synthetic sources. These materials may be used in a raw or refined state. Furthermore, they may be processed by the addition of a surfactant or by chemical reactions such as sulfonation, chlorination, and phosphation to produce a material that is emulsifiable in water. Tanned leathers do not usually contain sufficient lubricants, and for this reason fat liquoring is carried out in order to produce the desirable softness and pliability. The process involves oils or other materials penetrating the collagen structure of the leather, ideally so that each fiber becomes uniformly coated. The result improves not only the handle and feel of the leather, but also extensibility, tensile and tear strength, hydrophilic and hydrophobic properties and permeability to water and air.

It can be seen therefore that leather production is a complex process that involves the application of a variety of chemical materials, which, in general, must penetrate the collagen structure of the leather or at least become sufficiently chemically and/or physically bound. These agents frequently have to be employed at high concentrations, for long periods of time, and under particular processing conditions, such as high temperature, specific pH ranges and under particular mechanical conditions involving pressure or mechanical agitation.

These disadvantages can result in added expense due to the increased amounts of chemical materials required and due to processing power requirements. They can also result in environmental problems in removing and disposing of the excess of the treatment agents that are not permanently incorporated into the leather.

Textiles and leathers, which have a wide range of important uses in industry and society, are energy-intensive, water-intensive, and chemical-intensive to produce with several potential negative environmental and health implications. Accordingly, there is a need for improved compositions and methods for producing these goods.

### SUMMARY OF THE INVENTION

The subject invention provides environmentally-friendly compositions and methods for improving production of textiles and leathers. Advantageously, the compositions and methods can help reduce water and chemical usage resulting from these processes, as well as reduce wastewater pollution.

In certain embodiments, the methods of the subject invention comprise incorporating the application of a “green” molecule into a textile or leather-making process to reduce chemical usage, reduce water usage, reduce water pollution and/or provide an added benefit to the process. In certain embodiments, the methods of the subject invention comprise substituting a chemical surfactant with a “green” molecule in one or more steps involved in textile or leather-making process that would traditionally utilize a chemical surfactant.

In some embodiments, the “green” molecule is a biological amphiphilic molecule, which can be utilized as, for example, a detergent, a lubricant, an emulsifier, a wetting agent, a dispersant, an antimicrobial, a softening agent, or in other functions in the process of turning a raw material, e.g., a fiber or hide, into a finished product, e.g., a fabric or leather product.

Furthermore, in some embodiments, the biological amphiphilic molecule can be utilized as an adjuvant or additive for improving the performance of, for example, detergents, lubricants, dyes, finishing agents, antimicrobials, emulsifiers, softening agents, liming agents, de-liming agents, enzymes, tanning agents, oils, or other treatments utilized in textile and leather production.

In certain embodiments, green methods are provided for improving textile and/or leather production, wherein the method comprises obtaining a raw material, followed by applying a composition comprising one or more biological amphiphilic molecules to the raw material, wherein the biological amphiphilic molecule serves as an active ingredient and/or as an adjuvant or additive for an ingredient utilized in one or more steps involved with transforming the raw material into the textile or leather.

In certain embodiments, the raw material is a fiber, such as spun yarn or thread. In certain embodiments, the raw material is a sheet of fabric or cloth made from the weaving or knitting of yarn or thread. In certain embodiments, the raw material is a textile, such as clothing, drapes, or carpet. In certain other embodiments, the raw material is an animal hide or skin.

In certain embodiments, wherein the raw material is a fiber, fabric, cloth, or textile, the method comprises one or more of the following steps in the transforming of the raw material into a textile product: washing, desizing, scouring, lubricating, stonewashing, bleaching, dyeing, printing and/or finishing the raw material. In preferred embodiments, the biological amphiphilic molecule is used as an active ingredient and/or as an adjuvant or additive in one or more of these steps.

In certain embodiments, wherein the raw material is an animal hide or skin, the method comprises one or more of the following steps in the transforming of the raw material into a leather good: preserving, soaking, cleaning, degreasing, liming, de-liming, bating, pickling, bleaching, tanning, dyeing, drying, fatliquoring, softening and finishing. In preferred embodiments, the biological amphiphilic molecule is used as an active ingredient and/or as an adjuvant or additive in one or more of these steps.

In preferred embodiments, the biological amphiphilic molecule is a glycolipid biosurfactant (e.g., sophorolipids, rhamnolipids, cellobiose lipids, mannosylerythritol lipids and/or trehalose lipids). In some embodiments, other biosurfactants can be utilized, such as, for example, lipopeptides (e.g., surfactin, iturin, fengycin, arthrofactin and/or lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes.

In certain embodiments, the methods utilize a composition comprising one or more sophorolipid (SLP) molecules and/or a yeast culture comprising a SLP molecule. The SLP molecule can be, for example, an acidic (linear) SLP (ASL), lactonic SLP (LSL), di-acetylated SLP, mono-acetylated SLP, esterified SLP, amino-acid-SLP conjugate, metal-SLP conjugate, salt form SLP, SLP amino alcohols, SLP with carbonyl groups removed from the aliphatic chain, and/or any other derivatives of SLP molecules. The SLP molecule(s) can be in a pure form or crude form.

In certain embodiments, the present invention utilizes yeast strains and/or by-products of their growth. For example, in some embodiments, the methods comprise application of a microbe-based product comprising cultivated Starmerella bombicola ATCC 22214 and/or products of the growth of that microbe, such as SLP. In certain embodiments, the yeast in the composition can be inactive and/or in various growth states, such as, for example, vegetative or spore forms. In certain other embodiments, the yeast cells are removed from the culture so that broth, microbial growth by-products and, in some instances, small amounts of residual cellular matter remain for use.

Advantageously, SLP, when used according to the subject invention, have several benefits that make them ideal for use in the textile and leather making industries. First, their excellent wetting ability helps facilitate a reduction in the usage of water and chemical wetting agents; thus, they can contribute to reduced water pollution and wastewater treatment due to textile and leather making processes. Additionally, in their natural state, their mildly anionic properties make them compatible with natural and synthetic fibers, and most cationic softeners and conditioners. Furthermore, SLP are multifunctional, have a low critical micelle concentration (CMC), and are biodegradable.

### DETAILED DESCRIPTION

The subject invention provides environmentally-friendly compositions and methods for improving production of textiles and leather. Advantageously, the compositions and methods can help reduce water and chemical usage resulting from these processes, as well as reduce wastewater pollution.

In certain embodiments, the methods of the subject invention comprise incorporating the application of a biological amphiphilic molecule into a textile or leather-making process to reduce chemical usage, reduce water usage, reduce water pollution and/or provide an added benefit to the process. In certain embodiments, the methods of the subject invention comprise substituting a chemical surfactant with a biological amphiphilic molecule in one or more steps involved in textile or leather-making process that would traditionally utilize a chemical surfactant.

### Selected Definitions

As used herein, a “green” compound or material means at least 95% derived from natural, biological and/or renewable sources, such as plants, animals, minerals and/or microorganisms, and furthermore, the compound or material is biodegradable. Additionally, “green” compounds or materials are minimally toxic to humans and have a LD50>5000 mg/kg. A “green” product preferably does not contain any of the following: non-plant based ethoxylated surfactants, linear alkylbenzene sulfonates (LAS), ether sulfates surfactants or nonylphenol ethoxylate (NPE).

As used herein, a “biofilm” is a complex aggregate of microorganisms, such as bacteria, yeast, or fungi, wherein the cells adhere to each other and/or to a surface using an extracellular matrix. The cells in biofilms are physiologically distinct from planktonic cells of the same organism, which are single cells that can float or swim in liquid medium.

As used herein, “finishing” in the context of textile production and leather production means application of a process, chemical or substance to the textile or leather to enhance the performance or appearance thereof and/or ultimately convert it into a usable product. Finishing of textiles can include, for example, washing, bleaching, dyeing, conditioning, coating, glazing, polishing, calendaring, shrinking, raising, peaching, embellishing, or applying an antimicrobial, fire retardant, odor blocker, UV blocker, softener, or wrinkle guard. Finishing of leather goods can include, for example, washing, bleaching, dyeing, coating, drying, glazing, polishing, oiling (e.g., fatliquoring), or applying a coating, such as resins, lacquers, acrylics or polyurethanes.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An isolated microbial strain means that the strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 98%, by weight the compound of interest. For example, a purified compound is one that is at least 80%, 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism or a substance necessary for taking part in a particular metabolic process. A metabolite can be an organic compound that is a starting material, an intermediate in, or an end product of metabolism. Examples of metabolites include, but are not limited to, enzymes, acids, solvents, alcohols, proteins, vitamins, minerals, microelements, amino acids, biopolymers and biosurfactants.

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. The microbes may be present in or removed from the composition. The microbes can be present, with broth in which they were grown, in the microbe-based composition. The cells may be present at, for example, a concentration of at least 1×10³, 1×10⁴, 1×10¹, 1×10⁶, 1×10⁷, 1×10⁸, 1×10⁹, 1×10¹⁰, 1×10¹¹, 1×10¹², 1×10¹³ or more CFU per milliliter of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, carriers, such as water, salt solutions, or any other appropriate carrier, added nutrients to support further microbial growth, non-nutrient growth enhancers, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, a “raw material” includes any basic material from which a product is made. In certain embodiments, the raw material has been unaltered from its natural state. In certain embodiments, the raw material has been treated in some way, for example, in a previous step as part of a process. Thus, the raw material can be a starting material, and/or it can be an intermediary material in a process.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

As used herein, “surfactant” means a compound that lowers the surface tension (or interfacial tension) between two liquids or between a liquid and a solid. Surfactants act as, e.g., detergents, wetting agents, emulsifiers, foaming agents, and/or dispersants. A “biosurfactant” is a surface-active substance produced by a living cell and/or using naturally-derived substrates.

Biosurfactants are a structurally diverse group of surface-active substances consisting of two parts: a polar (hydrophilic) moiety and non-polar (hydrophobic) group. Due to their amphiphilic structure, biosurfactants can, for example, increase the surface area of hydrophobic water-insoluble substances, increase the water bioavailability of such substances, and change the properties of bacterial cell surfaces. Biosurfactants can also reduce the interfacial tension between water and oil and, therefore, lower the hydrostatic pressure required to move entrapped liquid to overcome the capillary effect. Biosurfactants accumulate at interfaces, thus reducing interfacial tension and leading to the formation of aggregated micellar structures in solution. The formation of micelles provides a physical mechanism to mobilize, for example, oil in a moving aqueous phase.

The ability of biosurfactants to form pores and destabilize biological membranes also permits their use as antibacterial, antifungal, and hemolytic agents to, for example, control pests and/or microbial growth.

Typically, the hydrophilic group of a biosurfactant is a sugar (e.g., a mono-, di-, or polysaccharide) or a peptide, while the hydrophobic group is typically a fatty acid. Thus, there are countless potential variations of biosurfactant molecules based on, for example, type of sugar, number of sugars, size of peptides, which amino acids are present in the peptides, fatty acid length, saturation of fatty acids, additional acetylation, additional functional groups, esterification, polarity and charge of the molecule.

These variations lead to a group of molecules comprising a wide variety of classes, including, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes. Each type of biosurfactant within each class can further comprise subtypes having further modified structures.

Like chemical surfactants, each biosurfactant molecule has its own HLB value depending on its structure; however, unlike production of chemical surfactants, which results in a single molecule with a single HLB value or range, one cycle of biosurfactant production typically results in a mixture of biosurfactant molecules (e.g., subtypes and isomers thereof).

The phrases “biosurfactant” and “biosurfactant molecule” include all forms, analogs, orthologs, isomers, and natural and/or anthropogenic modifications of any biosurfactant class (e.g., glycolipid) and/or subtype thereof (e.g., sophorolipid).

As used herein, the term “sophorolipid,” “sophorolipid molecule,” “SLP” or “SLP molecule” includes all forms, and isomers thereof, of SLP molecules, including, for example, acidic (linear) SLP (ASL) and lactonic SLP (LSL). Further included are mono-acetylated SLP, di-acetylated SLP, esterified SLP, SLP with varying hydrophobic chain lengths, cationic and/or anionic SLP with fatty acid-amino acid complexes attached, esterified SLP, SLP-metal complexes, salt form SLP, SLP amino alcohols, SLP with carbonyl groups removed from the aliphatic chain, and other, including those that are and/or are not described within in this disclosure.

In preferred embodiments, the SLP molecules according to the subject invention are represented by General Formula (1) and/or General Formula (2) and are obtained as a collection of 30 or more types of structural homologues having different fatty acid chain lengths (R³), and, in some instances, having an acetylation or protonation at R¹ and/or R².

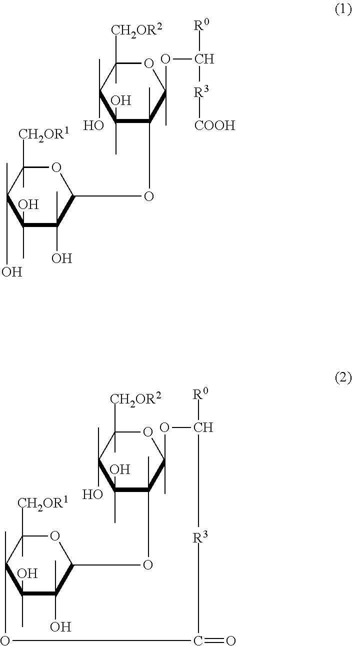

In General Formula (1) or (2), R⁰ can be either a hydrogen atom or a methyl group. R¹ and R² are each independently a hydrogen atom or an acetyl group. R³ is a saturated aliphatic hydrocarbon chain, or an unsaturated aliphatic hydrocarbon chain having at least one double bond, and may have one or more Substituents.

Examples of the Substituents include halogen atoms, hydroxyl, lower (C1-6) alkyl groups, halo lower (C1-6) alkyl groups, hydroxy lower (C1-6) alkyl groups, halo lower (C1-6) alkoxy groups, amino acid residues, and others. R³ typically has 11 to 20 carbon atoms. In certain embodiments of the subject invention, R³ has 9-18 carbon atoms.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Methods for Improving Textile Processing and Leather Making

The subject invention provides environmentally-friendly compositions and methods for improving production of textiles and leathers. Advantageously, the compositions and methods can help reduce water and chemical usage resulting from these processes, as well as reduce wastewater pollution.

In certain embodiments, the methods of the subject invention comprise incorporating the application of a “green” molecule into a textile or leather-making process to reduce chemical usage, reduce water usage, reduce water pollution and/or provide an added benefit to the process. In certain embodiments, the methods of the subject invention comprise substituting a chemical surfactant with a “green” molecule in one or more steps involved in textile or leather-making process that would traditionally utilize a chemical surfactant.

In some embodiments, the “green” molecule is a biological amphiphilic molecule, which can be utilized as, for example, a detergent, a lubricant, an emulsifier, a wetting agent, a dispersant, an antimicrobial, a softening agent or conditioner, or in other functions in the process of turning a raw material, e.g., a fiber or hide, into a finished product, e.g., a fabric or leather product.

Furthermore, in some embodiments, the biological amphiphilic molecule can be utilized as an adjuvant or additive for improving the performance of, for example, detergents, lubricants, dyes, finishing agents, antimicrobials, emulsifiers, softening agents, liming agents, de-liming agents, tanning agents, oils, or other treatments utilized in textile and leather production.

In certain embodiments, methods are provided for improving the environmental impact of textile and/or leather production, wherein the method comprises obtaining a raw material, followed by applying a composition comprising one or more biological amphiphilic molecules to the raw material, wherein the biological amphiphilic molecule serves as an active ingredient and/or as an adjuvant or additive for an ingredient utilized in one or more steps involved with transforming the raw material into the textile or leather.

In certain embodiments, the raw material is a fiber, such as spun yarn or thread. In certain embodiments, the raw material is a sheet of fabric or cloth made from the weaving or knitting of yarn or thread. In certain embodiments, the raw material is a textile, such as clothing, drapes, or carpet. In certain other embodiments, the raw material is an animal hide or skin.

### Textiles

In general, “textile” refers to any flexible material, such as a fabric, cloth or carpet, created by interlocking yarns or threads, which are produced by spinning raw fibers into long and twisted lengths. The interlocking can be achieved via, for example, weaving, knitting, crocheting, knotting, tatting, felting, bonding or braiding. In addition to the flexible material, “textile” as used herein can also include the finished products created using the flexible material, as well as the raw materials involved in producing the flexible materials, including the raw fibers, yarns and threads. In certain embodiments, finished textiles include clothing, upholstery, drapes, carpets and rugs. In certain embodiments, textiles can also include paper products.

Textiles can be made from, for example, protein-rich sources (e.g., wool, silk, hair, fur), cellulose-rich sources (e.g., cotton, flax, hemp, coconut, wood, and other plants), minerals (e.g., asbestos), as well as synthetic sources (e.g., polyamines, polyesters, acrylonitriles, polyurethanes, and other polymers).

In certain embodiments, the subject method comprises one or more of the following steps in the transforming of the raw material into a finished textile product: washing, desizing, scouring, lubricating, stonewashing, bleaching, dyeing, printing and/or finishing a textile raw material. In preferred embodiments, a biological amphiphilic molecule is used as an active ingredient and/or as an adjuvant or additive in one or more of these steps.

Because of the water- and chemical-intensive nature of many of these steps, the biological amphiphilic molecule is advantageous in that it can help facilitate the reduction in water and chemical usage.

In a specific exemplary embodiment, the biological amphiphilic molecule serves as a detergent in the washing of the raw material to remove dirt and other contaminants.

In another specific exemplary embodiment the biological amphiphilic molecule serves as a scouring agent to remove impurities and increase the hydrophilicity and absorbency of the raw material. Scouring typically involves the saponification of fats using a caustic agent, emulsification of waxes, and/or removal and dispersion of dust and dirt particles using a detergent.

In certain embodiments, the biological amphiphilic molecule can be useful in reducing water and chemical usage by improving one or more aspects of scouring. For example, the biological amphiphilic molecule can enhance the effects of a caustic saponification agent, thereby reducing the amount required for solubilizing fats. Alternatively, the biological amphiphilic molecule can serve as a replacement for the caustic agent, wherein the fats are effectively sequestered and/or dispersed by the biological amphiphilic molecule. Additionally, the biological amphiphilic molecule can serve as an oil-in-water emulsifier for solubilizing and emulsifying waxy impurities. Furthermore, as noted previously, the biological amphiphilic molecule can serve as a detergent for removing and dispersing dust and dirt particles.

Advantageously, through use of the biological amphiphilic molecule, traditional scouring compounds, such as silicates, phosphates, linear alkyl benzene sulfonates, APEO/NPEO, fatty alcohol sulfates, and ethoxylated alcohols can be reduced and/or replaced with a low-foaming, high wettability, biodegradable, non-toxic alternative.

In another exemplary embodiment the biological amphiphilic molecule serves as an emulsifier for spin finishes, which lubricate and soften the raw material to prevent breakage, dryness, static and/or stiffness after scouring. Spin finishes include lubricants such as, e.g., mineral oils, waxes and oils of esters, as well as polyalkenylene oxides, polyalkenylene and silicon oils. Emulsifiers are necessary for keeping these lubricating materials dispersed, as well as for the purpose of washing out the spin finish. Emulsifiers include, e.g., ethers and soaps of fatty acids, ethers of fatty alcohols, fatty amino ethers, and sulfates. Accordingly, through the use of the biological amphiphilic molecule, traditional emulsifiers can be reduced and/or replaced with a green alternative.

In yet another exemplary embodiment, the biological amphiphilic molecule serves as a wetting agent to prepare the raw material to receive dyes, inks and/or finishing treatments and enhance their penetration and binding to the raw material. The biological amphiphilic molecule can also serve as a dispersing adjuvant to improve the spread and uniformity of dye, ink and/or finishing treatment particles throughout the raw material.

In yet another exemplary embodiment, the biological amphiphilic molecule serves as an active ingredient and/or as an adjuvant during finishing of the raw material. For example, the biological amphiphilic molecule can serve as an antimicrobial treatment, a softening treatment, and/or as a wetting agent or additive to enhance the penetration and binding of fire retardants, antimicrobials, softeners, polishing agents, enzymes, shrinking agents and antistatic agents.

Wetting agents are important components that make textile fibers more susceptible to water immersion and, thus, more efficiently treated with water-based compounds. Standard wetting agents in the textile industry include, for example, alkyl sulfate, sulfonate, fatty acid or fatty acid ester sulfate, carboxylic acid soap, phosphate ester, polyoxyethylene alkyl phenol ether, polyoxyethylene aliphatic alcohol ether, polyoxyethylene polyoxyethylene propylene block copolymer and others. Accordingly, through the use of the biological amphiphilic molecule, traditional chemical wetting agents can be reduced and/or replaced with a green alternative.

In certain embodiments, the subject invention provides textiles and finished leather goods produced according to the subject methods. For example, in some embodiments, yarns, threads, fabrics, cloths, and/or carpets that are suitable for producing a finished textile good are provided, wherein the yarns, threads, fabrics, cloths, and/or carpets are impregnated with and/or coated with a formulation comprising a biological amphiphilic molecule according to the subject invention. Further provided are finished textile goods produced using the yarns, threads, fabrics, cloths, and/or carpets comprising the biological amphiphilic molecule. The finished product may comprise, for example, at least 0.0001%, 0.001%, 0.01%, 0.1%, 1%, 2%, 5% or more, by weight, of the biological amphiphilic molecule.

### Leather

In general, “leather” refers to a strong, flexible material obtained from chemical treatment of animal skins and hides to prevent decay. The most common leathers come from cattle, sheep, goats, equine animals, buffalo, pigs, and aquatic animals such as seals and alligators. As used herein, “leather” includes the flexible materials, as well as the raw materials for producing leathers, such as the skins or hides, and the finished products thereof. Finished leather products can include, for example, clothing, shoes, handbags, furniture, tools and sports equipment.

In certain embodiments, the subject method comprises one or more of the following steps in the transforming of leather raw materials in into a finished leather good: preserving, soaking, cleaning, degreasing, liming, pickling, bateing, tanning, dyeing, drying, fatliquoring, softening and finishing. In preferred embodiments, the biological amphiphilic molecule is used as an active ingredient and/or as an adjuvant or additive in one or more of these steps.

Because of the water- and chemical-intensive nature of many of these steps, the biological amphiphilic molecule is advantageous in that it can help facilitate a reduction in water and chemical usage.

In an exemplary embodiment, the biological amphiphilic molecule serves as an active ingredient and/or as an adjuvant in the preservation of the raw material after removal from an animal. In one embodiment, the biological amphiphilic molecule is an antimicrobial active ingredient to prevent the growth of decomposing microbes. In one embodiment, the biological amphiphilic molecule enhances the dispersion and penetration of salt and other preservatives (e.g., inorganic salts, sodium chloride and sodium sulfate) into the raw material. Accordingly, through use of the biological amphiphilic molecule, less salt is required for desired performance, and thus less water pollution with these inorganic substances.

In another exemplary embodiment the biological amphiphilic molecule serves as a detergent and/or degreaser for the cleaning of salts, dirt, flesh, wool, hair, grease and fats from a preserved hide or skin.

In another exemplary embodiment, the biological amphiphilic molecule can enhance the re-wetting of the raw material after preservation. In some embodiments, this involves adding the biological amphiphilic molecule to water and soaking the raw material therein for a period of time.

In another specific exemplary embodiment, the biological amphiphilic molecule serves as an adjuvant during alkali soaking, or liming, of the raw material, wherein the biological amphiphilic molecule accelerates the effects of the alkali solution in removing hair and scales, denaturing interfibrillary proteins, and otherwise swelling or “opening up” the raw material for tanning and finishing. Examples of alkali solutions include sodium hydroxide, calcium hydroxide, lime, sodium sulfide, sodium hydrosulfide, caustic soda, and soda ash.

In another exemplary embodiment, the biological amphiphilic molecule serves as an adjuvant during de-liming of the raw material, wherein the biological amphiphilic molecule accelerates the effects of agents used in removing liming agents. Examples of de-liming agents include formic acid, ammonium sulfate, ammonium chloride, sodium bisulfite, and sodium metabisulfiate. Accordingly, through the use of the biological amphiphilic molecule, use of traditional liming and de-liming agents can be reduced, along with the resulting effluent pollution.

In another exemplary embodiment, the biological amphiphilic molecule serves as an adjuvant during bating of the raw material. Bating involved the treatment of hides and skins with enzymes, such as digestive or pancreatic enzymes, which are used to remove residual skin, hair and scale components broken down by the liming process. The bating process can provide a smoother grain and rendering the hide soft and flexible. By applying the bating enzymes with a biological amphiphilic molecule of the subject invention, the subject method can enhance penetration of the enzymes into the raw material for more thorough and uniform enzymatic activity.

In another specific exemplary embodiment, the biological amphiphilic molecule serves as an adjuvant for tanning of the raw material, wherein the biological amphiphilic molecule enhances the penetration of the tanning agent into the raw materials to achieve more efficient tanning. Tanning is the process that converts the protein of the raw hide or skin into a stable material that will not putrefy and is suitable for a wide variety of end applications. Metal salts such as chromium salts, aluminum salts or zirconium salts or mixtures thereof are often use in tanning. Others include aldehydes, such as formaldehyde and/or glutaraldehyde, vegetable tannins, and zeolite. In some instances, sodium formate and other auxiliary chemicals are used during the tanning process to assist with the penetration of tanning agents. Accordingly, through the use of the biological amphiphilic molecule, use of traditional tanning agents and auxiliary chemicals can be reduced, along with resulting effluent pollution.

In yet another exemplary embodiment, the biological amphiphilic molecule serves as a wetting agent to prepare the raw material to receive dyes, inks and/or finishing treatments and enhance their penetration and binding to the raw material. The biological amphiphilic molecule can also serve as a dispersing adjuvant to improve the spread and uniformity of dye, ink and/or finishing treatment particles throughout the raw material.

In yet another exemplary embodiment, the biological amphiphilic molecule serves as an oil-in-water emulsifier for oils, fats and waxes used to treat leather for softness, pliability, hydrophilicity and/or hydrophobicity (i.e., fat liquoring). Additionally, the biological amphiphilic molecule can enhance the penetration of these lipid substances into the collagen structure of the leather so that each fiber becomes uniformly coated.

In yet another exemplary embodiment, the biological amphiphilic molecule serves as an active ingredient and/or as an adjuvant during finishing of the raw material. For example, the biological amphiphilic molecule can serve as an antimicrobial treatment, a softening treatment, and/or as an additive to enhance the adhesion, penetration and/or dispersion of resins, lacquers, acrylics or polyurethane on the surface of the raw material. In further embodiments, the biological amphiphilic molecule can serve as an emulsifier, defoamer, and/or viscosity modifier in a finishing mixture.

In certain embodiments, the subject invention provides leather and finished leather goods produced according to the subject methods. For example, in some embodiments, leather that is suitable for producing a finished good is provided, wherein the leather is impregnated with and/or coated with a formulation comprising a biological amphiphilic molecule according to the subject invention. Further provided are finished leather goods produced using the leather comprising the biological amphiphilic molecule.

### Biological Amphiphilic Molecules

In certain embodiments, the biological amphiphilic molecule used according to the subject methods is a biosurfactant, meaning a surface-active compound that is produced by a cell and/or produced using naturally-derived substrates. In preferred embodiments, the biosurfactant is produced by a microorganism.

In preferred embodiments, the methods utilize a glycolipid biosurfactant (e.g., sophorolipids, rhamnolipids, cellobiose lipids, mannosylerythritol lipids and/or trehalose lipids). In some embodiments, other biosurfactants can be utilized, such as, for example, lipopeptides (e.g., surfactin, iturin, fengycin, arthrofactin and/or lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes.

In certain embodiments, the methods utilize a composition comprising one or more sophorolipid (SLP) molecules and/or a yeast culture comprising a SLP molecule. The SLP molecule can be, for example, an acidic (linear) SLP (ASL), lactonic SLP (LSL), di-acetylated SLP, mono-acetylated SLP, esterified SLP, amino-acid-SLP conjugate, metal-SLP conjugate, salt form SLP, SLP amino alcohols, SLP with carbonyl groups removed from the aliphatic chain, and/or any other derivatives of SLP molecules. The SLP molecule(s) can be in a pure form or crude form.

In certain embodiments, the present invention utilizes yeast strains and/or byproducts of their growth. For example, a microbe-based product comprising cultivated Starmerella bombicola ATCC 22214 and/or products of the growth of that microbe, such as SLP, can be used. In certain embodiments, the yeast in the composition can be inactive and/or in various growth states, such as, for example, vegetative or spore forms. In certain other embodiments, the yeast cells are removed from the culture so that broth, microbial growth by-products and, in some instances, trace amounts of residual inactive cellular matter remain for use.

Advantageously, SLP have several benefits making them well-suited for use in the textile and leather making industries. First, their excellent wetting ability helps facilitate a reduction in the usage of water and chemicals, they contribute to reduced water pollution and wastewater treatment due to textile and leather making processes. Additionally, in their natural state, their mildly anionic properties make them compatible with natural and synthetic fibers, and most cationic softeners. Furthermore, SLP are multifunctional, require a low critical micelle concentration (CMC), and are biodegradable.

In preferred embodiments, the subject invention provides methods for producing a “green” surfactant composition having one or more desired functional properties, the methods comprising identifying a biosurfactant molecule having a specific functional property and producing the biosurfactant molecule by cultivating a biosurfactant-producing microorganism under conditions favorable for production of the biosurfactant. In some embodiments, the method further comprises derivatizing the biosurfactant molecule, for example, to increase pH and temperature stability, or to impart or remove a net charge.

In certain embodiments, the method further comprises combining the biosurfactant molecule with one or more additional biosurfactant molecules, the identity, ratio and/or molecular structure of which are determined based on the desired use(s) for the composition. Thus, a composition is produced having one or more desired functional characteristics, including, for example, surface/interfacial tension reduction, viscosity reduction, emulsification, demulsification, solvency, detergency, and/or anti-microbial action.

In some embodiments, the identity, ratio and/or molecular structure of biosurfactant molecules in the green surfactant composition is determined based on, e.g., HLB, CMC, and/or KB, of the individual molecules. In some embodiments, the identity, ratio and/or molecular structure of biosurfactant molecules is determined based on a theoretical or actual desired HLB, CMC, and/or KB value for the composition as a whole.

The one or more biosurfactants can be produced using small to large scale cultivation methods. Most notably, the methods can be scaled to an industrial scale, i.e., a scale that is suitable for use in supplying biosurfactants in amounts to meet the demand for commercial applications, for example, production of compositions for enhanced oil recovery. In preferred embodiments, the biosurfactants are produced, optionally modified, and mixed at a centralized location that is, in some embodiments, not more than 300 miles, 200 miles, 100 miles, or 10 miles from where the green surfactant composition will be used.

The microorganisms utilized for producing the biosurfactants may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In certain embodiments, the microorganisms are bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquefaciens, B. circulans, B. firmus, B. laterosporus, B. licheniformis, B. megaterium, B. mojavensis, B. mucilaginosus, B. subtilis), Burkholderia (e.g., B. thailandensis), Frateuria (e.g., F. aurantia), Microbacterium (e.g., M. laevaniformans), myxobacteria (e.g., Myxococcus xanthus, Stignatella aurantiaca, Sorangium cellulosum, Minicystis rosea), Paenibacillus polymyxa, Pantoea (e.g., P. agglomerans), Pseudomonas (e.g., P. aeruginosa, P. chlororaphis subsp. aureofaciens (Kluyver), P. putida), Rhizobium spp., Rhodospirillum (e.g., R. rubrum), Sphingomonas (e.g., S. paucimobilis), and/or Thiobacillus thiooxidans (Acidothiobacillus thiooxidans).

In certain embodiments, the microorganism is a yeast or fungus. Yeast and fungus species suitable for use according to the current invention, include Aureobasidium (e.g., A. pullulans), Blakeslea, Candida (e.g., C. apicola, C. bombicola, C. nodaensis), Cryptococcus, Debaryomyces (e.g., D. hansenii), Entomophthora, Hanseniaspora, (e.g., H. uvarum), Hansenula, Issatchenkia, Kluyveromyces (e.g., K. phaffii), Mortierella, Mycorrhiza, Meyerozyma guilliermondii, Penicillium, Phycomyces, Pichia (e.g., P. anomala, P. guilliermondii, P. occidentalis, P. kudriavzevii), Pleurotus spp. (e.g., P. ostreatus), Pseudozyma (e.g., P. aphidis), Saccharomyces (e.g., S. boulardii sequela, S. cerevisiae, S. torula), Starmerella (e.g., S. bombicola), Torulopsis, Trichoderma (e.g., T. reesei, T. harzianum, T. hamatum, T. viride), Ustilago (e.g., U. maydis), Wickerhamomyces (e.g., W. anomalus), Williopsis (e.g., W. mrakii), Zygosaccharomyces (e.g., Z. bailii), and others.

In preferred embodiments, the microorganism is a yeast or fungus selected from: Starmerella spp. yeasts and/or Candida spp. yeasts, e.g., Starmerella (Candida) bombicola, Candida apicola, Candida batistae, Candida floricola, Candida riodocensis, Candida stellate and/or Candida kuoi. In a specific embodiment, the microorganism is Starmerella bombicola, e.g., strain ATCC 22214.

As used herein “fermentation” refers to growth or cultivation of cells under controlled conditions. The growth could be aerobic or anaerobic. Unless the context requires otherwise, the phrase is intended to encompass both the growth phase and product biosynthesis phase of the process.

As used herein, a “broth,” “culture broth,” or “fermentation broth” refers to a culture medium comprising at least nutrients. If the broth is referred to after a fermentation process, the broth may comprise microbial growth byproducts and/or microbial cells as well.

The microbe growth vessel used according to the subject invention can be any fermenter or cultivation reactor for industrial use. As used herein, the term “reactor,” “bioreactor,” “fermentation reactor” or “fermentation vessel” includes a fermentation device consisting of one or more vessels and/or towers or piping arrangements. Examples of such reactor includes, but are not limited to, the Continuous Stirred Tank Reactor (CSTR), Immobilized Cell Reactor (ICR), Trickle Bed Reactor (TBR), Bubble Colunm, Gas Lift Fermenter, Static Mixer, or other vessel or other device suitable for gas-liquid contact. In some embodiments, the bioreactor may comprise a first growth reactor and a second fermentation reactor. As such, when referring to the addition of substrate to the bioreactor or fermentation reaction, it should be understood to include addition to either or both of these reactors where appropriate.

In one embodiment, the method comprises inoculating a fermentation reactor comprising a liquid growth medium with a biosurfactant-producing microorganism to produce a culture; and cultivating the culture under conditions favorable for production of the biosurfactant.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, samples may be taken from the vessel for enumeration, purity measurements, biosurfactant concentration, and/or visible oil level monitoring. For example, in one embodiment, sampling can occur every 24 hours.

The microbial inoculant according to the subject methods preferably comprises cells and/or propagules of the desired microorganism, which can be prepared using any known fermentation method. The inoculant can be pre-mixed with water and/or a liquid growth medium, if desired.

In certain embodiments, the cultivation method utilizes submerged fermentation in a liquid growth medium. In one embodiment, the liquid growth medium comprises a carbon source. The carbon source can be a carbohydrate, such as glucose, dextrose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as canola oil, soybean oil, rice bran oil, olive oil, corn oil, sunflower oil, sesame oil, and/or linseed oil; powdered molasses, etc. These carbon sources may be used independently or in a combination of two or more. In preferred embodiments, a hydrophilic carbon source, e.g., glucose, and a hydrophobic carbon source, e.g., oil or fatty acids, are used.

In one embodiment, the liquid growth medium comprises a nitrogen source. The nitrogen source can be, for example, yeast extract, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid growth medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, proteins and microelements can be included, for example, corn flour, peptone, yeast extract, potato extract, beef extract, soybean extract, banana peel extract, and the like, or in purified forms. Amino acids such as, for example, those useful for biosynthesis of proteins, can also be included.

The method of cultivation can further provide oxygenation to the growing culture. One embodiment utilizes slow motion of air to remove low-oxygen containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid. In certain embodiments, dissolved oxygen (DO) levels are maintained at about 25% to about 75%, about 30% to about 70%, about 35% to about 65%, about 40% to about 60%, or about 50% of air saturation.

In some embodiments, the method for cultivation may further comprise adding additional acids and/or antimicrobials in the liquid medium before and/or during the cultivation process. Antimicrobial agents or antibiotics (e.g., streptomycin, oxytetracycline) are used for protecting the culture against contamination. In some embodiments, however, the metabolites produced by the yeast culture provide sufficient antimicrobial effects to prevent contamination of the culture.

In one embodiment, prior to inoculation, the components of the liquid culture medium can optionally be sterilized. In one embodiment, sterilization of the liquid growth medium can be achieved by placing the components of the liquid culture medium in water at a temperature of about 85-100° C. In one embodiment, sterilization can be achieved by dissolving the components in 1 to 3% hydrogen peroxide in a ratio of 1:3 (w/v).

In one embodiment, the equipment used for cultivation is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Gaskets, openings, tubing and other equipment parts can be sprayed with, for example, isopropyl alcohol. Air can be sterilized by methods know in the art. For example, the ambient air can pass through at least one filter before being introduced into the vessel. In other embodiments, the medium may be pasteurized or, optionally, no heat at all added, where the use of pH and/or low water activity may be exploited to control unwanted microbial growth.

The pH of the culture should be suitable for the microorganism of interest and can be altered as desired in order to produce a specific biosurfactant molecule in the culture. Buffers, and pH regulators, such as carbonates and phosphates, may be used to stabilize pH near a preferred value.

In some embodiments, the pH is about 2.0 to about 7.0. In some embodiments, the pH is about 2.5 to about 5.5, about 3.0 to about 4.5, or about 3.5 to about 4.0. In one embodiment, the cultivation may be carried out continuously at a constant pH. In another embodiment, the cultivation may be subject to changing pH.

In one embodiment, the method of cultivation is carried out at about 5° to about 100° C., about 150 to about 60° C., about 200 to about 45° C., about 22° to about 30° C., or about 240 to about 28° C. In one embodiment, the cultivation may be carried out continuously at a constant temperature. In another embodiment, the cultivation may be subject to changing temperatures.

According to the subject methods, the microorganisms can be incubated in the fermentation system for a time period sufficient to achieve a desired effect, e.g., production of a desired amount of cell biomass or a desired amount of one or more microbial growth by-products. The microbial growth by-product(s) produced by microorganisms may be retained in the microorganisms and/or secreted into the growth medium. The biomass content may be, for example from 5 g/l to 180 g/l or more, or from 10 g/l to 150 g/l.

In certain embodiments, fermentation of the culture occurs for about 48 to 150 hours, or about 72 to 150 hours, or about 96 to about 125 hours, or about 110 to about 120 hours.

After the fermentation cycle is complete, the method can comprise, in some embodiments, extracting, concentrating and/or purifying the biosurfactant molecule.

In certain embodiments, the methods of the subject invention can be carried out in such a way that minimal-to-zero waste products are produced, thereby reducing the amount of fermentation waste being drained into sewage and wastewater systems, and/or being disposed of in landfills.

The cell biomass collected from the culture after extraction of the biosurfactant would typically be inactivated and disposed of. However, the subject methods can further comprise collecting the cell biomass and using it, in live or inactive form, for a variety of purposes, including but not limited to, as a soil amendment, a livestock feed supplement, an oil well treatment, and/or a skincare product. The cell biomass can be used directly, or it can be mixed with additives specific for the intended use.

In some embodiments, water or other non-toxic liquids used to extract and/or purify the biosurfactant can contain residual biosurfactants, nutrients and/or cell matter. Thus, in certain embodiments, the liquids can be used in irrigation drip lines or sprinklers as a soil or foliar treatment for plants; as a safe nutritional and/or hydration supplement for humans and animals; as a cleaning composition; and/or for countless other uses to reduce fermentation waste products.

In some embodiments, the method comprises modifying the structure of a biosurfactant molecule prior to adding it to the composition.

In some embodiments, adjusting the parameters of fermentation results in modification and/or production of one or more specific biosurfactant molecules in the culture, and/or production of a specific ratio of multiple biosurfactant molecules. These parameters can include, for example, using a specific strain of microorganism, adjusting the growth medium composition, co-cultivating the microbe with an antagonistic and/or influencing microbe, adding inhibitors and/or stimulant compounds to the nutrient medium, adjusting the temperature, pH and/or aeration of fermentation, and others.

In some embodiments, the biosurfactant molecule(s) obtained from the fermentation cycle can be modified post-fermentation by, for example, esterification, polymerization, addition of amino acids, addition of metals, addition of amino alcohols, reduction of carbonyl groups, and alteration of fatty acid chain lengths.

In additional and/or alternative embodiments, the composition can be tailored to have a specific, and in some instances, very precise, HLB value based on the identity and ratio of biosurfactant molecules within the composition.

In certain embodiments, the composition comprises one or more biosurfactant molecules belonging to a class selected from, for example, glycolipids, lipopeptides, flavolipids, phospholipids, fatty acid ester compounds, lipoproteins, lipopolysaccharide-protein complexes, and polysaccharide-protein-fatty acid complexes.

In some embodiments, the composition comprises multiple biosurfactant molecules belonging to the same biosurfactant class. In some embodiments, the composition comprises biosurfactant molecules belonging to more than one of these biosurfactant classes.

In some embodiments, the composition comprises a glycolipid, such as, for example, a sophorolipid, rhamnolipid, trehalose lipid, cellobiose lipid and/or mannosylerythritol lipid.

In a specific embodiment, the composition can comprise 0% to 100%, 5% to 95%, 10% to 90%, 15% to 85%, 20% to 80%, 25% to 75%, 30% to 70%, 35% to 65%, 40% to 60%, 45% to 55%, or 50%, by weight, a sophorolipid molecule as defined elsewhere herein.

In a specific embodiment, the composition can comprise 0% to 100%, 5% to 95%, 10% to 90%, 15% to 85%, 20% to 80%, 25% to 75%, 30% to 70%, 35% to 65%, 40% to 60%, 45% to 55%, or 50%, by weight, a rhamnolipid molecule. A “rhamnolipid” or a “rhamnolipid molecule” can include, for example, mono- and di-rhamnolipids, and all possible derivatives therein, as well as other forms as described herein.

In a specific embodiment, the composition can comprise 0% to 100%, 5% to 95%, 10% to 90%, 15% to 85%, 20% to 80%, 25% to 75%, 30% to 70%, 35% to 65%, 40% to 60%, 45% to 55%, or 50%, by weight, a mannosylerythritol lipid molecule. A “mannosylerythritol lipid” or a “mannosylerythritol lipid molecule” can include, for example, tri-acylated, di-acylated, mono-acylated, tri-acetylated, di-acetylated, mono-acetylated and non-acetylated MEL, as well as stereoisomers and/or constitutional isomers thereof. In certain specific embodiments, the MEL are characterized as groups: MEL A (di-acetylated), MEL B (mono-acetylated at C4), MEL C (mono-acetylated at C6), MEL D (non-acetylated), tri-acetylated MEL A, tri-acetylated MEL B/C, as well as other forms as described herein.

In some embodiments, the composition comprises 0% to 100%, 5% to 95%, 10% to 90%, 15% to 85%, 20% to 80%, 25% to 75%, 30% to 70%, 35% to 65%, 40% to 60%, 45% to 55%, or 50%, by weight, a lipopeptide, such as, for example, a surfactin, fengycin, arthrofactin, lichenysin, iturin and/or viscosin.

In some embodiments, two or more purified biosurfactant molecules are mixed with one another. In some embodiments, two or more unpurified, or crude form, biosurfactants are mixed with one another, wherein the crude form can comprise, for example, residual nutrient medium, microbial cells, and/or other microbial metabolites produced during fermentation. In some embodiments, a purified biosurfactant molecule can be mixed with a crude form biosurfactant.

### Preparation of Microbe-Based Products

One microbe-based product of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based product may also comprise the broth and/or growth by-products with the microbes removed therefrom.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, ultra-violet light resistant agents, other microbes and other suitable additives that are customarily used for such preparations.

In one embodiment, buffering agents including organic and amino acids or their salts, can be added. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

The pH of the microbe-based composition should be suitable for the microorganism(s) of interest. In some embodiments, the pH of the composition is about 3.5 to 7.0, about 4.0 to 6.5, or about 5.0.

In one embodiment, additional components such as an aqueous preparation of a salt, such as sodium bicarbonate or carbonate, sodium sulfate, sodium phosphate, sodium biphosphate, can be included in the formulation.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used. For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules (e.g., spores), or a mixture of vegetative cells and propagules.

In one embodiment, the microbe growth facility is located on, or near, a site where the microbe-based products will be used, for example, within 300 miles, 200 miles, or even within 100 miles. Advantageously, this allows for the compositions to be tailored for use at a specified location. The formula and potency of microbe-based compositions can be customized for a specific application and in accordance with the local conditions at the time of application.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### Replacing Chemical Surfactants

In preferred embodiments, the subject green surfactant composition can be utilized in place of chemical surfactant(s) in products that would typically comprise the chemical surfactant(s), where one or more biosurfactants are chosen that have the same or similar functional properties as the chemical surfactant(s).

Thus, in some embodiments, the methods comprise selecting a known composition comprising one or more chemical surfactants and, optionally, one or more additional components, and producing an environmentally-friendly version of the known composition by using a green surfactant composition of the subject invention in place of the chemical surfactant(s). The green surfactant composition can be mixed with the one or more optional additional components, if present.

In certain embodiments, the compositions can be used to replace compositions comprising chemical surfactants. Typical chemical or synthetic surfactants (meaning, non-biological surfactants) comprise a hydrophobic group, which is usually a long hydrocarbon chain (C8-C18) that may or may not be branched, while the hydrophilic group is formed by moieties such as carboxylates, sulfates, sulfonates (anionic), alcohols, polyoxyethylenated chains (nonionic) and quaternary ammonium salts (cationic).

Non-biological surfactants that can be replaced in surfactant compositions utilizing the methods and compositions of the subject invention include, but are not limited to: anionic surfactants, ammonium lauryl sulfate, sodium lauryl sulfate (also called SDS, sodium dodecyl sulfate), alkyl-ether sulfates sodium laureth sulfate (also known as sodium lauryl ether sulfate (SLES)), sodium myreth sulfate; docusates, dioctyl sodium sulfosuccinate, perfluorooctanesulfonate (PFOS), perfluorobutanesulfonate, linear alkylbenzene sulfonates (LABs), alkyl-aryl ether phosphates, alkyl ether phosphate; carboxylates, alkyl carboxylates (soaps), sodium stearate, sodium lauroyl sarcosinate, carboxylate-based fluorosurfactants, perfluorononanoate, perfluorooctanoate; cationic surfactants, pH-dependent primary, secondary, or tertiary amines, octenidine dihydrochloride, permanently charged quaternary ammonium cations, alkyltrimethylammonium salts, cetyl trimethylammonium bromide (CTAB) (a.k.a. hexadecyl trimethyl ammonium bromide), cetyl trimethylammonium chloride (CTAC), cetylpyridinium chloride (CPC), benzalkonium chloride (BAC), benzethonium chloride (BZT), 5-Bromo-5-nitro-1,3-dioxane, dimethyldioctadecylammonium chloride, cetrimonium bromide, dioctadecyldi-methylammonium bromide (DODAB); zwitterionic (amphoteric) surfactants, sultaines CH APS (3-[(3-Cholamidopropyl)dimethylammonio]-1-propanesulfonate), cocamidopropyl hydroxysultaine, betaines, cocamidopropyl betaine, phosphatidylserine, phosphatidylethanolamine, phosphatidylcholine, sphingomyelins; nonionic surfactants, ethoxylate, long chain alcohols, fatty alcohols, cetyl alcohol, stearyl alcohol, cetostearyl alcohol, oleyl alcohol, polyoxyethylene glycol alkyl ethers (Brij): CH3-(CH2)10-16-(O—C2H4)1-25-OH (octaethylene glycol monododecyl ether, pentaethylene glycol monododecyl ether), polyoxypropylene glycol alkyl ethers: CH3-(CH2)10-16-(O—C3H6)1-25-OH, glucoside alkyl ethers: CH3-(CH2)10-16-(O-Glucoside)1-3-OH (decyl glucoside, lauryl glucoside, octyl glucoside), polyoxyethylene glycol octylphenol ethers: C8H17-(C6H4)-(O—C2H4)1-25-OH (Triton X-100), polyoxyethylene glycol alkylphenol ethers: C9H19-(C6H4)-(O—C2H4)1-25-OH (nonoxynol-9), glycerol alkyl esters (glyceryl laurate), polyoxyethylene glycol sorbitan alkyl esters (polysorbate), sorbitan alkyl esters (spans), cocamide MEA, cocamide DEA, dodecyldimethylamine oxide, copolymers of polyethylene glycol and polypropylene glycol (poloxamers), and polyethoxylated tallow amine (POEA).

Anionic surfactants contain anionic functional groups at their head, such as sulfate, sulfonate, phosphate, and carboxylates. Prominent alkyl sulfates include ammonium lauryl sulfate, sodium lauryl sulfate (also called SDS, sodium dodecyl sulfate) and the related alkyl-ether sulfates sodium laureth sulfate, also known as sodium lauryl ether sulfate (SLES), and sodium myreth sulfate. Carboxylates are the most common surfactants and comprise the alkyl carboxylates (soaps), such as sodium stearate.

Surfactants with cationic head groups include: pH-dependent primary, secondary, or tertiary amines; octenidine dihydrochloride; permanently charged quaternary ammonium cations such as alkyltrimethylammonium salts: cetyl trimethylammonium bromide (CTAB) a.k.a. hexadecyl trimethyl ammonium bromide, cetyl trimethylammonium chloride (CTAC); cetylpyridinium chloride (CPC); benzalkonium chloride (BAC); benzethonium chloride (BZT); 5-Bromo-5-nitro-1,3-dioxane; dimethyldioctadecylammonium chloride; cetrimonium bromide; and dioctadecyldi-methylammonium bromide (DODAB).

Zwitterionic (amphoteric) surfactants have both cationic and anionic centers attached to the same molecule. The cationic part is based on primary, secondary, or tertiary amines or quaternary ammonium cations. The anionic part can be more variable and include sulfonates. Zwitterionic surfactants commonly have a phosphate anion with an amine or ammonium, such as is found in the phospholipids phosphatidylserine, phosphatidylethanolamine, phosphatidylcholine, and sphingomyelins.

A surfactant with a non-charged hydrophilic part, e.g., ethoxylate, is non-ionic. Many long chain alcohols exhibit some surfactant properties.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Sophorolipid Production

To produce SLP, a fermentation reactor is inoculated with a Starmerella bombicola yeast. The temperature of fermentation is held at 23 to 28° C. After about 22 to 26 hours, the pH of the culture is set to about 3.0 to 4.0, or about 3.5, using 20% NaOH. The fermentation reactor comprises a computer that monitors the pH and controls the pump used to administer the base, so that the pH remains at 3.5.

After about 6-7 days of cultivation (120 hours+/−1 hour), if 7.5 ml of a SLP layer is visible with no oil visible and no glucose detected, the batch is ready for harvesting.

### Modifying SLP Products During Fermentation

The structure of the SLP molecules produced by the subject methods can be modified in multiple ways by altering fermentation parameters. One approach is to include long-chain fatty alcohols (e.g., C₄ to C₂₆-alcohols) in the nutrient medium. The resulting SLP molecules will comprise hydrophobic moieties up to C₃₆ in length, and will increase the hydrophobicity, emulsification and detergency capabilities of the composition.

Another approach is to limit the amount of sugar and/or oil in the fermentation medium. For example, in some embodiments, the amount of glucose is limited to about 25 g/L to about 75 g/L and/or the amount of canola oil is limited to about 25 ml/L to about 75 ml/L. In certain embodiments, this will increase the amount of ASL produced in the culture.

To increase the amount of hydrophobic SLP molecules (e.g., LSL and some ASL) the yeast is cultivated at a temperature of about 22° C. to about 28° C., and at a pH of about 2.5 to 4.0, where the pH begins at about 4.0 and reduces to—and is stabilized at—about 2.5 during cultivation.

To increase the amount of ASL in the culture, the yeast is cultivated at a pH of about 5.5, and at a temperature of about 35° C. Additionally, utilizing the yeast Candida kuoi can result in a composition comprising only ASL, as this yeast only produces ASL.

### Modifying SLP Products After Fermentation

Some modifications of SLP molecules occur after the cultivation cycle is ended. For example, inorganic acids, alkaline substances and/or salts can be mixed with SLP to alter solubility.

Furthermore, in addition to SLP, the yeasts also produce enzymes, such as lipases and esterases, into the yeast culture. Certain enzymes catalyze the bonding of amino acids to the SLP molecules. Thus, amino acids can be added to the yeast culture, and are chosen based on the character of the amino acid and the desired character of the SLP molecule(s). Cationic, anionic, polar and non-polar amino acids and amino alcohols, when bonded to the SLP molecules, can alter the properties of the SLP molecules to be, for example, cationic, anionic, polar or non-polar. This can also be achieved using synthetic means.

Additionally, certain enzymes catalyze the esterification of the SLP molecules in the presence of the alcohol and fatty acid.

When the fermentation cycle is completed, an alcohol (e.g., 10% v/v) selected from methanol, ethanol, isopropyl alcohol, hexanol, or heptanol is added to the yeast culture. The liquid fermentation medium preferably already comprises a source of fatty acids, for example, canola oil. However, additional fatty acids can be added if a certain esterified product is desired, for example, purified forms of fatty acids such as palmitic, stearic, oleic, linoleic, linolenic, ricinoleic, lauric, and myristic acids.

The yeast culture with alcohol and fatty acid is mixed for 24 hours. After 24 hours, mixing is stopped and the culture will contain SLP esters containing an added alcohol, a sophorose, and a fatty acid ester, e.g., methanol sophorolipid oleic acid ester, which is formed when methanol and oleic acid are used.

We claim:

1. A method for producing a textile or leather product, the method comprising applying a biological amphiphilic molecule to a raw material, wherein the biological amphiphilic molecule serves as an adjuvant, an additive and/or an active ingredient in transforming the raw material into the textile or leather product.

2. The method of claim 1, wherein the biological amphiphilic molecule functions as a detergent, a lubricant, an emulsifier, a wetting agent, a dispersant, an antimicrobial, and/or a softening agent.

3. The method of claim 1, wherein the biological amphiphilic molecule functions as an adjuvant or additive for improving the performance of detergents, lubricants, dyes, finishing agents, antimicrobials, emulsifiers, softening agents, liming agents, tanning agents, oils, or other treatments utilized in textile and leather production.

4. The method of claim 1, wherein the raw material is a fiber, fabric, cloth, or textile, and wherein the method comprises applying the biological amphiphilic molecule in one or more of the following steps involved in transforming of the raw material into a finished textile product: washing, desizing, scouring, lubricating, stonewashing, bleaching, dyeing, printing and/or finishing the raw material.

5. The method of claim 1, wherein the raw material is an animal hide or skin, and wherein the method comprises applying the biological amphiphilic molecule in one or more of the following steps in transforming of the raw material into a finished leather good: preserving, soaking, cleaning, degreasing, liming, tanning, dyeing, drying, softening and finishing.

6. The method of claim 1, wherein the biological amphiphilic molecule is a biosurfactant.

7. The method of claim 6, wherein the biosurfactant is a glycolipid biosurfactant selected from sophorolipids, rhamnolipids, cellobiose lipids, mannosylerythritol lipids and trehalose lipids, a lipopeptide selected from surfactin, iturin, fengycin, arthrofactin and lichenysin, a flavolipid, a phospholipid, a fatty acid ester, or a high molecular weight polymer selected from lipoproteins, lipopolysaccharide-protein complexes, and polysaccharide-protein-fatty acid complexes.

8. The method of claim 6, wherein the biosurfactant is a sophorolipid.

9. The method of claim 8, wherein the biosurfactant is in a purified form.

10. The method of claim 8, wherein the biosurfactant was produced by fermentation of Starmerella bombicola, and wherein the method comprises applying the biosurfactant in the form of a broth resulting from the fermentation.

11. The method of claim 10, wherein the broth comprises yeast cell matter.

12. The method of claim 1, wherein water usage is reduced as a result of application of the biological amphiphilic molecule.

13. The method of claim 1, wherein chemical usage is reduced as a result of application of the biological amphiphilic molecule.

14. The method of claim 1, wherein water pollution is reduced as a result of application of the biological amphiphilic molecule.

15. A textile good comprising yarn, thread, fabric, cloth, or a finished product produced from yard, thread, fabric, and/or cloth, wherein the yarn, thread, fabric, cloth is impregnated with and/or coated with a biological amphiphilic molecule.

16. The textile good of claim 15, wherein the biological amphiphilic molecule is a sophorolipid.

17. The textile good of claim 15, wherein the finished product is selected from clothing, upholstery, drapes, carpets and rugs.

18. A leather good comprising an animal hide or skin, or a finished product produced from an animal hide or skin, wherein the animal hide or skin is impregnated with and/or coated with a biological amphiphilic molecule.

19. The leather good of claim 18, wherein the biological amphiphilic molecule is a sophorolipid.

20. The leather good of claim 18, wherein the finished product is selected from clothing, upholstery, handbags, shoes, and sports equipment.
